(19) United States
(12) Patent Application Publication
(10) Pub. No.: US 2009/0204338 A1
(43) Pub. Date: Aug. 13, 2009

(54) Method of deriving a quantitative measure of the instability of calcific deposits of a blood vessel

(75) Inventors: Mads Nielsen, Dragor (DK); Francois B. Lauze, Vanlose (DK); Marleen De Bruijne, Copenhagen (DK); Erik B. Dam, Copenhagen (DK); Morten A. Karsdal, Copenhagen (DK); Claus Christiansen, Morcote (CH)

Correspondence Address: Edwards Angell Palmer & Dodge LLP, P.O.Box 55874, Boston, MA, 02205, US

(73) Assignee: Nordic Bioscience A/S

(21) Appl. No.: 12/069,894

(22) Filed: Feb. 13, 2008

(51) Int. Cl. G01N 33/48

(52) U.S. Cl. 702/19

(57) ABSTRACT

A computer-implemented method of processing an image of at least part of a blood vessel to derive a measure indicative of the instability of calcific deposits in the blood vessel, said blood vessel containing at least one calcific deposit, comprises locating and annotating one or more calcific deposits. Using information derived from the annotation of said calcific deposits, the method further comprises calculating a measure reflecting either one or both of a) the aggregate of the deviations from roundness of individual calcific deposits, and b) up to at least a threshold value, the extent to which the separate calcific deposits are spaced from one another.

### FIELD OF INVENTION

The present invention relates to a method of deriving a quantitative measure of the instability of calcific deposits of a blood vessel.

### BACKGROUND OF THE INVENTION

Cardiovascular disease (CVD) is at present the most common cause of death in the developed world and almost one million deaths are caused by CVD annually. Despite vast epidemiologic and interventional studies that demonstrate significant declines in CVD incidence and prevalence with adherence to a healthy lifestyle and identification and treatment of risk factors, CVD mortality remains significant. Two thirds of women who die suddenly of CVD have no previously recognised symptoms.

An overwhelming range of potential risk factors for assessing CVD risk have already been identified. It is therefore unlikely that identification of additional independent risk factors will adequately identify patients at risk, because the more dominant factors are likely to have already been identified. For this reason, several multivariate analysis models have been suggested for estimation of risk in populations. For example, the SCORE (Systemic Coronary Risk Evaluation) system has been devised to provide a standard method of assessing risk of CVD. This system has been developed to define the lifestyle, risk factor and therapeutic targets for CVD prevention.

However, the SCORE system and other similar systems that have been devised all rely on the collection of a number of independent variables associated with a person, for example, age, sex, smoking habits, weight, height etc, that are then computed to assess risk of CVD. These methods do not involve considering the physical state of the cardiovascular system itself.

Since all major risk factors appear to have been identified, the focus has shifted to further understanding, analysis, automation and simplification of the dominant risk factors. Currently a large amount of interest relating to aortic calcifications has been devoted to findings relating to heritage, coronary calcifications, clinical vascular disease, cholesterol, and depression. Various studies have shown a correlation between aortic and coronary calcium. In type II diabetes patients, it has been shown that aortic calcification is an independent risk factor of clinical vascular disease. From all of these studies, it is clear that aortic calcification is an important factor of cardiovascular disease.

Kauppila et al. (Kauppila, Polak, Cupples, Hannan, Kiel, Wilson “New indices to classify location, severity and progression of calcific lesions in the abdominal aorta: a 25-year follow-up study”) describes a segment-wise scoring system to determine the extent of calcification of an aorta. The most common of their scoring systems is referred to as the aortic calcification severity scoring system “AC24”. For purposes of the AC24 scoring system, a lumbar radiograph image of an artery is split into eight segments according to the position of the four lumbar vertebrae, L1 to L4, and the anterior and posterior wall as shown in FIG. 1. Each segment is given a value of 0 to 3, according to the quantity of calcium visible in that segment. Specifically, 0 implies no aortic calcific deposits; 1—small scattered calcific deposits filling less than ⅓ of the longitudinal wall of the aorta; 2—⅓ or more, but less than ⅔ of the longitudinal wall of the aorta are calcified; 3—⅔ or more of the longitudinal wall is calcified. For the AC24 score the scores of the individual areas for both the posterior and anterior wall are summed.

The AC24 scoring system purports to provide a simple, low cost assessment of subclinical vascular disease. The division into segments has several advantages as the segmentation approach will only produce a large score when the calcified plaque is distributed over the full lumbar aorta. However, the method is still heavily reliant on the observations of a clinician when grading the different segments of the aorta. Furthermore, the method does not discriminate between severity and spread of individual calcifications. In this respect, a similar score may be returned in the situation where either one segment is severely calcified or several segments are slightly calcified.

### SUMMARY OF THE INVENTION

According to a first aspect of the present invention there is provided a computer-implemented method of processing an image of at least part of a blood vessel to derive a measure indicative of the instability of calcific deposits in the blood vessel, said blood vessel containing at least one calcific deposit, which method comprises locating and annotating one or more calcific deposits, using information derived from the annotation of said calcific deposits to calculate a measure reflecting either one or both of a) the aggregate of the deviations from roundness of individual calcific deposits, and b) up to at least a threshold value, the extent to which the separate calcific deposits are spaced from one another.

The present inventors have found that, in biological terms, a greater number of small calcific deposits distributed over a large portion of a blood vessel indicate a greater risk of developing cardiovascular disease than fewer larger deposits over the same area. The inventors have also found that the risk that a patient may suffer an episode of cardiovascular disease is high while calcific deposits are growing as during growth, the calcific deposits are relatively unstable. Because of its size relative to the size of a blood vessel a big, dense calcific deposit might be thought to be of grave concern, but could be quite stable and safe in terms of resulting in an episode of cardiovascular disease. By contrast, several small calcific deposits might seem not to be severe because of their size, but may carry a greater risk of resulting in an episode of cardiovascular disease as they would be associated with a greater risk of growth. The scope for growth of individual calcifications also increases as the periphery of a calcification becomes more irregular and deviates from being round.

By way of the method defined above, the present inventors take into consideration one or both of the spread of calcific deposits in a blood vessel and the scope of growth of the deposits to provide an indication of how stable the calcifications are.

This contrasts with the AC24 system in which an equal score may be returned for a given number of equi-sized calcifications irrespective of whether or not they are adjacent one another or spread along the aorta and in which no account is taken of the shape of the deposits.

In a preferred embodiment, locating and annotating the one or more calcific deposits comprises locating and annotating the boundary of each said calcific deposit and calculating a measure reflecting a combination of a) and b) is obtained by calculating the area occupied by the calcific deposits, expanding the boundary of each said calcific deposit outwards by a distance x corresponding to between 4 mm and 20 mm of a life-size image, calculating the area occupied by the expanded calcific deposits, and calculating a comparative index by comparing the area of expanded calcific deposits with the area of unexpanded calcific deposits to derive said measure.

Expanding the boundaries of calcific deposits in the blood vessel gives an indication of how the individual calcific deposits may grow. In this respect, expansion of the boundaries of the calcific deposits can portray molecular action that precedes the formation of calcific deposits but that is unseen in an image of a blood vessel. By expanding the boundaries of the individual calcifications by a fixed distance, any calcific deposits that lie within close proximity and at least within the fixed distance of each other will expand into each other. When calculating the measure, overlapping areas of adjacent expanded calcific deposits will only be considered once. Thus, the measure is able to reflect how, in reality, the calcific deposits may grow.

Preferably, the method further comprises counting the number of calcific deposits and weighting said comparative index by said number. Weighting the number of calcifications with the comparison between the area of expanded calcific deposits and the area of unexpanded calcific deposits provides an enhanced measure of the degree of calcification in a blood vessel and the associated risk of developing cardiovascular disease.

Preferably, the distance x of expansion of the boundaries corresponds to between 7 mm and 10 mm of a life-size image. More preferably, the distance x corresponds to approximately 8.9 mm of a life-size image.

The typical diameter of a healthy aorta is approximately 20 mm-25 mm. The typical diameter of a diseased aorta may be up to 60 mm-65 mm. In an embodiment, boundaries of the calcifications are expanded by approximately ⅙ to ½ of the diameter of the aorta.

Preferably, the step of expanding the boundary of the one or more calcific deposits comprises dilating the boundary of each calcific deposit.

In embodiments, the boundaries of the calcific deposits may be dilated using any suitable structuring element resulting in expansion of the boundaries by the approximate distance x.

For example, the boundaries of the one or more calcific deposits may be dilated using a square of side length 2x. In a preferred embodiment, the boundaries of the one or more calcific deposits are dilated using a circle of radius x.

In an embodiment, points along the boundary of each respective calcific deposit are moved outwards by the fixed distance x or, if closer, up to an aortic wall or unexpanded boundary of an adjacent calcific deposit. Preventing expansion of the boundaries of respective areas of calcification beyond either the arterial walls or adjacent calcific deposits gives a realistic prediction of the likely growth of the calcific deposits.

Additionally and/or alternatively, the method for calculating a measure reflecting b) comprises identifying a convex hull of the calcific deposits and deriving a value representative of the convex hull by calculating one of the perimeter of the convex hull and the area within the convex hull.

The convex hull of the calcific deposits defines the shortest path around the calcific deposits that encloses each of the calcific deposits. The convex hull increases as the calcific deposits are more spread out throughout the blood vessel.

Preferably, the method further comprises calculating a value indicative of the total area of the calcific deposits and dividing the value representative of the convex hull by the total area.

Alternatively and/or additionally, the method further comprises counting the number of calcific deposits and deriving a value indicative of the product obtained by multiplying the number of calcific deposits with the value representative of the convex hull.

In an alternative embodiment, the method for calculating a measure reflecting a) comprises identifying a convex hull of each individual calcific deposit, deriving a value representative of each convex hull by calculating one of the perimeter of the convex hull and the area within the convex hull, summing the values representative of the convex hulls, calculating a value indicative of the total area of the calcific deposits and dividing the sum of values representative of the convex hulls by the total area of calcific deposits.

In an embodiment, calculating a measure reflecting a) comprises deriving a value indicative of the result of calculating the ratio of the square of the perimeter to the area for each calcific deposit and summing the ratios.

In an alternative embodiment, calculating a measure reflecting a) comprises deriving a value indicative of the result of calculating the ratio of the square of the sum of the perimeters of the calcific deposits to the sum of the areas of the calcific deposits.

As stated above, the present inventors have found that the rate of growth of individual calcifications is likely to increase as the periphery of the individual calcific deposits become more irregular and as the individual calcific deposits deviate from being round. As the calcific deposits deviate from being round and as the periphery becomes more irregular the ratio of perimeter to area increases.

Additionally and/or alternatively, calculating a measure reflecting a) or b) further comprises calculating a value indicative of a fractal dimension of the calcific deposits.

Preferably, the method further comprises calculating the Hausdorff Dimension or using a box-counting method to calculate the value indicative of the fractal dimension.

At coarser resolutions of grid when using the box-counting method, an indication of the spread of calcific deposits within an aorta is determined as the fractal dimension will increase as the calcific deposits are more spread out. If the grid is relatively large, a greater percentage of boxes of the grid will be occupied by at least some part of a calcific deposit if the calcific deposits are spread out.

At finer resolutions of grid when using the box-counting method, an indication of the irregularity of the periphery of individual calcifications can be determined and the fractal dimension will increase as the periphery of calcific deposits become more irregular. If the grid is relatively fine, a greater percentage of boxes of the grid will be occupied by at least some part of a calcific deposit if the periphery of the calcific deposit is irregular.

Additionally and/or alternatively, calculating a measure reflecting b) further comprises calculating a value indicative of the entropy of the calcific deposits.

If all calcific deposits are located in close proximity to one another, the entropy of the calcific deposits will return a lower score than if the calcific deposits are spread out throughout the blood vessel.

Alternatively and/or additionally, calculating a measure reflecting b) further comprises calculating a value indicative of the sum of the distances between calcific deposits.

Whilst the invention is applicable to any blood vessel, in a preferred embodiment the blood vessel is an artery and in a more preferred embodiment the blood vessel is an aorta.

In the above embodiments, a high score generally indicates a lack of stability of the calcifications and indicates a higher risk that a patient may suffer an episode of cardiovascular disease. It will, however, be appreciated that an inverse of the measure could be obtained, or other known mathematical techniques could be applied to the measure, such that a lower score would indicate a lack of stability.

Although the invention has principally been defined as a method of extracting significant information from a digital image, it is of course equally applicable as an instruction set for a computer for carrying out a said method or as a suitably programmed computer.

### BRIEF DESCRIPTION OF THE DRAWING

Embodiments of the present invention will hereinafter be described with reference to the accompanying drawings, in which:

FIG. 1 shows schematically a prior art scoring system for lumbar aortic calcification;

FIG. 2 shows an x-ray of a lumbar aorta with calcific deposits in the lower region;

FIG. 3 shows the x-ray of the lumbar aorta of FIG. 2 where the aorta and boundaries of the respective calcific deposits in the aorta have been annotated;

FIG. 4 shows an image of an aorta with the boundaries of the respective calcific deposits expanded in accordance with an example of an embodiment of the present invention;

FIG. 5 illustrates schematically dilation of a shape; and

FIG. 6 shows the odds ratio of death versus survival using different techniques.

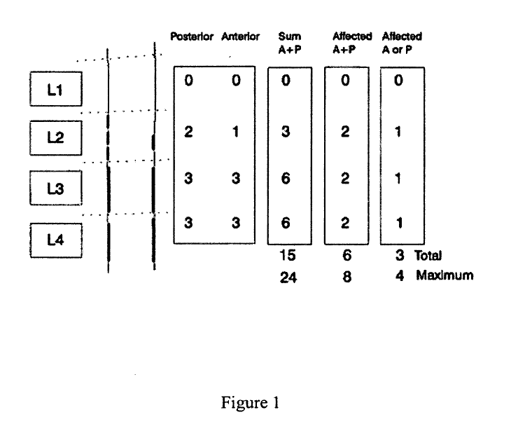

### DESCRIPTION OF THE PREFERRED EMBODIMENT

The present invention will hereinafter be described with particular reference to the analysis of x-ray images of an aorta. It will, however, be appreciated that the described method could be applied to other medical images of an aorta for example, DXA, Computer Tomography (CT) or Magnetic Resonance. Furthermore, the invention is not limited to analysis of images of an aorta and may also be applied to other blood vessels.

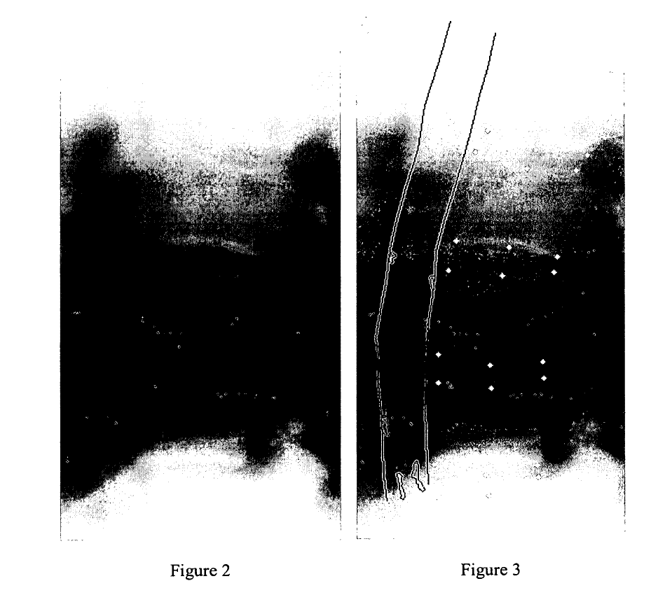

The first step in preparing the image for analysis is to outline the walls of the lumbar aorta in an image. FIG. 2 shows an image of part of a lumbar spine and lumbar aorta where there are calcific deposits 4 in the lumbar aorta. The six points for vertebral height measurements are annotated on L1 to L4 of the lumbar vertebrae as shown in FIG. 3 and from this the lumbar aorta can be identified and annotated. Further information about how the outline of the aorta is found is given by Lauze F et al. in (“Towards automated detection and segmentation of aortic calcifications from radiographs; proc of SPIE medical imaging 2007; 6512) and Conrad-Hansen et al. in (“Quantifying calcification in the lumbar aorta on x-ray images” in N. Ayache, S. Ourselin, and A. Maeder, editors; Medical Image Computing & Computer-Assisted Intervention; volume 4792 of Lecture Notes in Computer Science, pages 352-359, Springer, 2007).

The second step is to outline each individual calcific deposit located in the aorta. Annotation of the boundaries may be done manually or using a particle filtering technique as discussed by de Bruijne in (“Shape particle guided tissue classification” in Mathematical Methods in Biomedical Image Analysis (MMBIA), 2006) and Conrad-Hansen et al. in (“A pixelwise inpainting-based refinement scheme for quantizing calcification in the lumbar aorta on 2D lateral x-ray images”, SPIE Medical Imaging—Image Processing, 2006).

Based on the annotations of the calcific deposits, the following severity scores relating to the geometrical outline of the calcific deposits and aorta may be computed. In addition, using these annotations known calcification severity scores, for example the AC24, can also be calculated.
Area fraction (Area %)—the percentile of area of the projected lumbar (L1-L4) aorta covered by calcific deposits. Area percentage may relate to the surface area of the interface between the plaque and the lumen, giving more weight to the central part of the aorta, and this may indirectly relate to the risk of rupture.
Wall deposit thickness percentile (Thickness %)—the average thickness of calcific deposits along the aorta wall in relation to the aorta width. The width of the plaques located in the aorta may relate to the hydrodynamic resistance in the aorta and thereby to blood pressure known to be among the dominant risk factors for CVD.
Wall fraction (Wall%)—the percentile of lumbar (L1-L4) aorta wall covered by calcific deposits.
Length fraction (Length%)—the fraction of the length of the aorta where a calcific deposit is present at any position (anterior, posterior or internal). The length fraction of the lumbar aortic wall covered by arteriosclerosis may relate to the surface area of the interface between the plaque and the lumen, and thereby indirectly to the risk of rupture.
Number of calcific deposits (NCD)—the number of distinct calcific deposits in the lumbar region (L1-L4). The number of calcific deposits may relate to the number of independent pathologies with growth potential. Thus, many small calcifications may indicate many potential sources for progression.

In a preferred embodiment of the present invention, the annotations of the calcifications are used to calculate the following measures:
Morphological Artherosclerotic Distribution (MAD) factor—a measure of the total extent of the simulated atherosclerotic process divided by the area of the visible calcified plaques. The MAD factor provides a measure based on the area of all calcific deposits and takes into consideration the unseen molecular activity that precedes the formation of a calcification. The MAD factor therefore enables a measure that extends beyond the x-ray visible periphery of the calcification.

In summary, the total area of visible calcific deposits, i.e. the total area of calcification located within the respective annotated boundaries is calculated. The annotated boundary of each distinct calcific deposit is then expanded by a uniform amount and the total area of expanded calcific deposits, i.e. the total area of calcification located within the respective expanded annotated boundaries, calculated. The MAD factor is the result of the total expanded area of calcifications divided by the total area of visible (unexpanded) calcifications.

If points along the boundary of each calcific deposit are moved outwards from the centre of the calcific deposit by a distance x, the expanded boundaries of neighbouring calcific deposits or neighbouring portions of a particular calcific deposit that are within a distance x of each other will overlap. The MAD factor relates to the relative potential growth of the calcified plaque by counting only once overlapping expansions of two or more nearby calcific deposits. Likewise, expansion of the boundaries can be limited to expansion within the aortic walls. Therefore, if a calcific deposit is less than a distance x from an aortic wall, the boundary may only be expanded up to the aortic wall. This enables a measure to be derived that provides a realistic indication of the likely expansion of the calcific deposits.

Up to a certain threshold value, the MAD factor will in general be large when the individual areas are distributed over a large portion of the aorta. In this respect, relative proximity of the calcific deposits will only be considered if at least two of the calcific deposits are less than a distance x apart from each other. If the calcific deposits are all more than a distance x apart from each other, the expanded boundaries will not overlap.

The MAD factor also takes into account the morphology of individual calcific deposits. Specifically, as the shape of an individual calcific deposit deviates from being round, the percentage by which the calcified deposit will be expanded will be greater than for a rounder calcific deposit of the same area. Likewise, as the periphery of a calcific deposit becomes more irregular, the percentage by which the calcific deposit will expand will be greater than for a calcific deposit with a smoother periphery. Accordingly, a far stretched deposit yields a worse prognosis compared to a circular deposit of the same area.

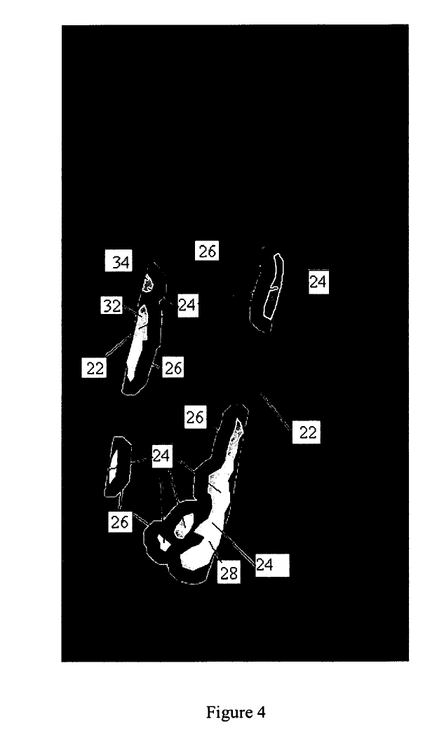

An example of an embodiment of the present invention illustrating the potential growth of areas of calcification is shown in FIG. 4. The first step is to locate the aortic walls 22 and annotate the boundaries of each area of calcification 24. FIG. 4 shows seven distinct areas of calcification 24. Points (not shown) along the respective boundary of each area of calcification are extended outwards in a direction substantially perpendicular to the tangent of each respective point. The points are moved a uniform distance away from the original boundary resulting in the expanded boundaries 26 shown in FIG. 4.

Expansion of the respective boundaries is restricted by the aortic walls and by other neighbouring calcifications. For example, as shown in FIG. 4, the boundary of a first calcification 28 is located in close proximity to an aortic wall 22. Accordingly, in that direction, the boundary is only expanded up to the aortic wall 22. Likewise, where calcifications 32, 34, are in close proximity to each other, the respective boundaries are only expanded up to the original unexpanded boundaries of the neighbouring calcifications and overlapping areas are only considered once.

In a specific embodiment a grass-fire equation implemented by iterated morphological dilations with a combined radius of 200 pixels corresponding to 8.9 mm in real size simulates the total extent of the atherosclerotic process. The boundaries may be expanded by the number of pixels that correspond to life-size distances of, for example, between 4 mm and 20 mm, or 7 mm and 10 mm. Typically, a healthy aorta has a diameter of approximately 20 mm to 25 mm. A diseased may have a diameter approximately 40 mm to 50 mm wider than a healthy aorta. Accordingly, boundaries of the calcifications may be expanded by approximately ⅙ to ½ of the diameter of the aorta. If the resolution of the image being analysed varies, the boundaries may be dilated by an appropriate number of pixels to correspond to an appropriate life-size expansion within the range specified above.

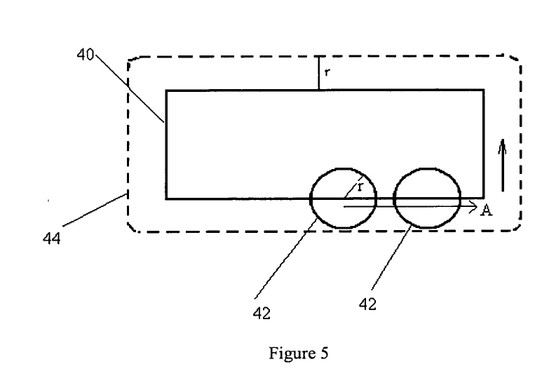

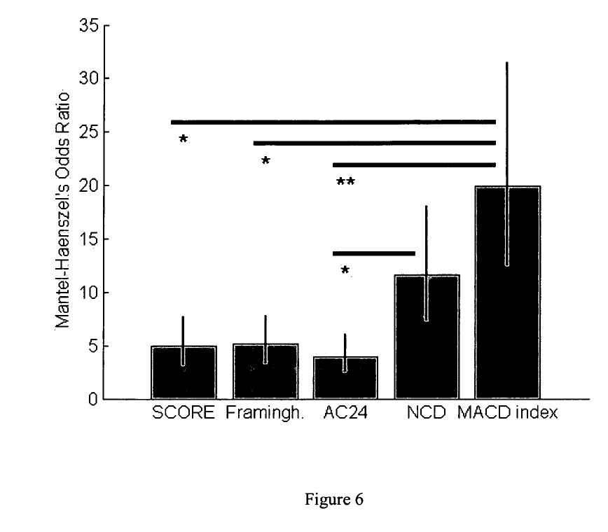

FIG. 5 illustrates schematically dilation of a general shape 40. To dilate the shape 40, the centre of, for example, a circle 42 having radius r is rolled along the periphery of the shape 40 in a direction A. The dilated shape 44 is determined by the circumference of the circle 42 and its periphery is a distance r away from that of the original shape 40.

This method can be performed digitally on, for example, the areas of calcification 24 shown in FIG. 4.

Alternatively, predicted growth of an area of calcification could be based on models of growth learned from previous examples of development of calcific deposits as set out by Kuhl, R Maas, G Himpel, A Menzel (“Computational modelling of artherosclerosis—A first approach towards a patient specific simulation based on computer topography”, BMMB 6, 321-331, 2007).
Morphological Atherosclerotic Calcification Distribution (MACD) index—the morphological atherosclerotic distribution (MAD) factor weighted by the number of calcified deposits (NCD). The MACD index takes into account both the number of calcific deposits and their relative growth by multiplying the MAD factor by the number of calcified deposits.

In isolation, the MAD factor does not account for multiple small calcific deposits that are spread out and where the expanded boundaries do not overlap. By including the number of calcific deposits in the calculation, an enhanced measure can be derived of the likely progression of calcification in a blood vessel and the associated risk of developing cardiovascular disease. In this respect, in biological terms, a greater number of small calcific deposits distributed over a large portion of a blood vessel indicates a greater risk of developing cardiovascular disease than fewer larger deposits over the same area.
Moment of inertia—the sum of an approximation of the amount of energy required to rotate each individual calcified pixel about a centre of mass. To calculate the moment of inertia it is first necessary to locate the overall centre of mass of the calcific deposits. The moment of inertia is equal to the sum of squared distances of each calcified pixel from the centre of mass. Accordingly, if a total of 100 calcified pixels are spread throughout an aorta in the form of many small calcified deposits, this will result in a higher moment of inertia than if the 100 calcified pixels form a single larger calcific deposit. The moment of inertia provides a measure of how spaced apart calcified pixels (and therefore calcified deposits) are, with little dependency on their shape.

To provide a more meaningful measure representative of the potential risk of progression of calcifications, the moment of inertia may be multiplied by a measure of the total area of calcific deposits or by the NCD.
Convex hull—the smallest convex set that contains all calcific deposits. The convex hull equates to the shortest path around the calcific deposits that encloses each of the calcific deposits. Calculating the perimeter of the convex hull or the area within the convex hull provides a measure of the spread of the calcific deposits. This value increases as the calcific deposits are more spread out. To obtain a more meaningful measure of the likely risk of developing cardiovascular disease, the convex hull may be multiplied by the number of calcific deposits or the total calcified area.

The convex hull of individual calcific deposits may also be calculated. The convex hull of an individual calcific deposit would equate to the shortest path around that calcific deposit and may therefore give some indication of the irregularity of individual calcific deposits. To provide a useful measure indicative of the aggregate deviation from roundness of calcific deposits, a measure may be derived of the sum of perimeters or areas representative of the convex hulls of individual calcific deposits divided by the total area of calcific deposits.
Shape index—a measure of the relationship between the perimeter and area of individual calcifications. In an embodiment, the aggregate shape index is derived by calculating the ratio of squared perimeter to area for each calcific deposit and summing the ratios:

$$Aggregate\_shape \_index = {\sum }_{n = 1}^{n = n} \frac{{p}_{n}^{2}}{{a}_{n}}$$

Alternatively, in a preferred embodiment, the aggregate shape index of all calcifications is calculated as the ratio of the squared sum of perimeters of the calcific deposits to the total area:

$$Aggregate\_shape \_index = \frac{{( {\sum }_{n = 1}^{n = n} {p}_{n} )}^{2}}{{\sum }_{n = 1}^{n = n} {a}_{n}}$$

As the perimeter of each of the calcific deposits increases compared to the respective areas, the shape index will increase. Accordingly, the shape index increases as the individual calcifications deviate from being round and as the respective peripheries increase in irregularity.
Fractal dimension—provides a measure of whether or not a pattern is space filling. At coarser resolution, an indication of the spread of calcific deposits within an aorta is determined as the fractal dimension will increase as the calcific deposits are more spread out. Using the box counting method, if the grid is relatively large, a greater percentage of boxes of the grid will be occupied by at least some part of a calcific deposit if the calcific deposits are spread out.

At finer resolution, an indication of the irregularity of the periphery of individual calcifications can be determined and the fractal dimension will increase as the periphery of calcific deposits become more irregular. Using the box counting method, if the grid is relatively fine, a greater percentage of boxes of the grid will be occupied by at least some part of a calcific deposit if the periphery of the calcific deposit is irregular.
Entropy—a measure of the disorder of calcified pixels in an aorta. At a given resolution, say 4×4 pixel grid, the number of calcified pixels in every 4×4 square is calculated. Assuming each 4×4 tile is identified by I and has a number of calcified pixels n(i), the total number of calcified pixels will be N=sum_i n(i). The probability of a calcified pixel belonging to the ith tile will then be p(i)=n(i)/N. The entropy of this distribution will be H=sum_i—p(i)log p(i). If all calcific deposits fall within a few tiles it will be a low number. If the calcific deposits are more spread out, the number will increase.
Quantification of distances between calcific deposits—a measure of the spacing between individual calcific deposits. As an example, this could be measured by making a minimal spanning tree of all calcific deposits using the standard Euclidean distance between the closest points of two calcific deposits. The aggregate distance between closest points of respective calcific deposits can be used to determine the spread of the calcific deposits in the aorta.

Each of the measures described above may be used in isolation to provide a measure indicative of the severity of calcification in an aorta. To verify results, however, or to provide repeatable results, the different methods may be used in combination.

Scores obtained for the various measures as they are described above are expected to increase as stability of the calcifications decreases and therefore the risk of suffering an episode of CVD increases. However, it will be appreciated that similarly useful results may be obtained with different mathematical formulae to obtain a result that may decrease or behave in a different way as stability of the calcifications in an aorta decreases.

Using the information derived above, the inventors have investigated whether information (e.g. number, length, width, morphology and patterns) harvested from aortic calcifications by automated image analysis could facilitate the identification of postmenopausal women at increased risk of accelerated arteriosclerosis and related adverse outcomes. It was further investigated whether generalised risk assessment techniques such as the SCORE card or Framingham point score or individual risk factors such as cholesterol or triglycerides levels, would bring additional information to advanced image analysis or arteriosclerotic calcifications by x-ray, in terms of prediction of CVD related deaths.

The calcification of aortic plaque is the end stage of a long range of molecular events resulting in maturation into a calcified fibro-fatty plaque that includes but is not restricted to: inflammation, macrophage infiltration, foam cells generation, lipid accumulation and processing and smooth muscle cells apoptosis. This results in imparted collagen synthesis and vascular integrity, and later results in weakened fibrous cap and generates atherosclerotic plaques that are more prone to rupture. Importantly, the calcifications detected and analysed on x-rays are restricted only to the calcified core, and do not include the surrounding necrotic tissue and area of high remodelling and fibrosis. Thus the pathological area is underestimated by simple calcification measurements on x-rays.

Hence, as described above, in a preferred embodiment the present inventors use area enhancement by mathematical modelling and pattern recognition, in which particular consideration of the plaque morphology and biology may be given to enable a measure of relative risk using traditional atherosclerotic scoring by the Framingham system, using for example, number, length, width, morphology and patterns.

A specific study using the MAD factor and other measures is described below. The study population consisted of 308 women aged 48 to 76 years who previously participated in epidemiologic cohorts. The original population was recruited by questionnaire. These women were invited for a follow up visit in 2000-2001. Among those 8593 women invited for a re-visit, 308 were randomly selected that all had an interval of 8-9 years since their first visit, were post menopausal, and had the lumbar aorta visible on a single radiograph in the examinations. Among these 308 women, 52 had died before the revisit. Of these 52, 20 died from CVD (38%), 27 died from cancer (52%) and 5 died from other causes (10%). Information of the 52 individuals who died in the observation period was obtained via the Central Registry of the Danish Ministry of Health with a follow up rate of 100%.

Demographic characteristics and risk parameters collected at baseline were age, weight, height, body mass index (BMI), waist and hip circumferences, systolic and diastolic blood pressure, treated hypertension, treated diabetes, smoking, regular alcohol and daily coffee consumption, and weekly fitness activity. Using a blood analyser, measurements of fasting glucose and lipid profile (total cholesterol, triglycerides, HDL0cholesterol (HDL0C), LDL-cholesterol (LDL-C), apolipoprotein (apoA and apoB) were obtained.

Lateral x-rays of the lumbar aorta (L1-L4) were recorded. The images were digitised using a Vidar Dosimetry Pro Advantage scanner providing an image resolution of 9651 times 4008 pixels on 12-bit gray scale using a pixel size of 44.6 μm squared. Trained radiologists annotated the digitised images on a Sectra radiological reading unit with annotation software written using the Matlab programming environment. The radiologists were instructed to annotate the 4 corner points and 2 mediolateral points used for vertebral height measurements on L1 to L4, then to delineate the aorta and finally to delineate every individual calcified deposit visible in the lumber aorta. The software used had the ability to edit annotations and to perform a digital zoom for precise annotation. Finally it was noted if the calcified deposit was associated to the anterior and/or posterior aorta wall.

Data presented is expressed as mean±SEM unless otherwise indicated. For comparison purposes, groups are adjusted for age, waist and triglyceride concentration. Differences are tested by a two-sided heteroscedastic student's t-test. Differences were considered statistically significant if p<0.05.

The comparison of markers was performed by adjusting one marker for the influence of the other. When the adjusted marker may significantly (p<0.05) differentiate survivor group from deceased group the marker is assumed to carry additional information. Markers are compared by mutually adjusting for the other marker and testing for additional information as above. Markers are furthermore compared by odds-ratio of the 90% fractile using the Mantel-Haenszel 95% confidence interval (Mantel N, Haenszel “Statistical aspects of the analysis of data from retrospective studies of disease” J National Cancer Inst 1959; 22(4):710-748). Odds ratio differences are tested by Tarone's (Tarone R E “On heterogenenity tests based on efficient scores” Biometrika 1985; 72(1):91-95) adjustment of the Breslow-Day (Breslow N E, Day N E “Statistical methods in cancer research. Volume I—the analysis of case-control studies” IARC Sci Publications 1980;(32):5-338) test of heterogeneous odds ratio. Markers are combined linearly using Fisher's linear discriminant analysis (LDA). When combining LDA with fractile analysis, the LDA weights and fractile threshold are computed and evaluated in a leave-one-out fashion. Tests are considered statistically significant when p<0.05.

Among the physical and metabolic markers, separation of survivors and deceased was provided by most markers: Age (p<0.001), Waist/Hip ratio (p=0.005), Systolic BP (p<0.001), Glucose (p=0.03), Cholesterol (p=0.006), triglycerides (p<0.001), and ApoB/ApoA (p=0.003). After adjustment by age, waist circumference, and triglyceride concentration, no metabolic or physical marker showed any predictive value of all-cause mortality, as shown below.

| | Population | Survivors | Deseased | | p-value |
|---|---|---|---|---|---|
| | (n = 308) | (n = 256) | (n = 52) | p-value | Adjusted |
| Age (years) | 60.3 ± 7.5 | 59.3 ± 7.1 | 65.6 ± 7.0 | <0.001 | — |
| Waist (cm) | 80.7 ± 10.9 | 80.2 ± 9.9 | 83.1 ± 12.4 | 0.07 | — |
| Waist-to-hip ratio | 0.80 ± 0.08 | 0.80 ± 0.08 | 0.83 ± 0.10 | 0.005 | — |
| BMI (kg/m²) | 24.7 ± 3.9 | 24.7 ± 3.8 | 25.1 ± 4.6 | 0.50 | 0.33 |
| Systolic BP (mm Hg) | 127 ± 21 | 125 ± 20 | 136 ± 26 | <0.001 | 0.39 |
| Diastolic BP (mm Hg) | 77 ± 10 | 76 ± 10 | 77 ± 11 | 0.52 | 0.87 |
| Hypertension % | 32 | 15 | 17 | 0.73 | 0.60 |
| Glucose (mmol/L) | 5.44 ± 1.27 | 5.37 ± 0.99 | 5.79 ± 2.17 | 0.03 | 0.44 |
| Total cholesterol (mmol/L) | 6.44 ± 1.19 | 6.36 ± 1.14 | 6.85 ± 1.33 | 0.006 | 0.96 |
| Triglyceride (mmol/L) | 1.24 ± 0.75 | 1.15 ± 0.56 | 1.69 ± 1.25 | <0.001 | — |
| LDL-C/mmol/L) | 2.89 ± 0.82 | 2.85 ± 0.80 | 3.07 ± 0.93 | 0.1 | 0.23 |
| HDL-C/mmol/L) | 1.77 ± 0.48 | 1.77 ± 0.44 | 1.74 ± 0.62 | 0.67 | 0.24 |
| ApoB/ApoA | 0.57 ± 0.18 | 0.56 ± 0.17 | 0.64 ± 0.23 | 0.003 | 0.59 |
| Lp(a) mg/dL | 21.4 ± 21.7 | 21.9 ± 22.0 | 18.4 ± 19.8 | 0.32 | 0.08 |

The aortic calcification markers performed significantly better in all the stratified deceased groups (except the other-cause death group), and unlike the metabolic/physical, all the aortic calcification markers scored significantly higher in the deceased versus the survivors, even after adjustment by age, waist and triglycerides.

| | | Deceased | Deceased | Deceased | Deceased | Deceased |
|---|---|---|---|---|---|---|
| Stratification/ | Survivors | CVD | Cancer | Other | CVD/Cancer | All cause |
| Method | (n = 256) | (n = 20) | (n = 27) | (n = 5) | (n = 47) | (n = 52) |
| AC24 | 1.35 ± 0.15 | 3.50 ± 0.12 | 3.42 ± 0.58 | 1.22 ± 0.98 | 3.45 ± 0.42 | 3.23 ± 0.40 |
| | | (0.03) | (0.004) | (0.64) | (0.002) | (0.006) |
| Area % | 0.53 ± 0.07 | 1.01 ± 0.04 | 1.58 ± 0.36 | 0.17 ± 0.15 | 1.34 ± 0.22 | 1.23 ± 0.21 |
| | | (0.60) | (0.005) | (0.30) | (0.04) | (0.04) |
| Thickness % | 8.9 ± 1.2 | 16.9 ± 0.8 | 25.0 ± 5.4 | 2.8 ± 2.6 | 21.6 ± 3.5 | 19.8 ± 3.3 |
| | | (0.66) | (0.01) | (0.29) | (0.03) | (0.07) |
| Wall % | 0.79 ± 0.10 | 2.08 ± 0.09 | 2.51 ± 0.52 | 0.60 ± 0.55 | 2.33 ± 0.34 | 2.16 ± 0.32 |
| | | (0.07) | (0.004) | (0.57) | (0.002) | (0.003) |
| Length % | 6.0 ± 0.7 | 15.3 ± 0.6 | 17.3 ± 3.4 | 4.8 ± 4.4 | 16.5 ± 2.2 | 15.4 ± 2.1 |
| | | (0.07) | (0.001) | (0.54) | (0.002) | (0.005) |
| NCD | 2.6 ± 0.4 | 8.5 ± 1.5 | 11.6 ± 2.6 | 3.0 ± 2.8 | 10.3 ± 1.6 | 9.6 ± 1.5 |
| | | (0.04) | (<0.001) | (0.84) | (<0.001) | (<0.001) |
| MAD factor | 1.26 ± 0.10 | 3.02 ± 0.27 | 2.25 ± 0.28 | 1.66 ± 1.14 | 2.58 ± 0.20 | 2.49 ± 0.21 |
| | | (0.002) | (0.17) | (0.78) | (0.004) | (0.004) |
| MACD | 1.91 ± 0.15 | 4.95 ± 0.43 | 4.03 ± 0.50 | 2.34 ± 1.61 | 4.42 ± 0.34 | 4.22 ± 0.34 |
| index | | (<0.001) | (0.01) | (0.94) | (<0.001) | (<0.001) |

The differences were larger comparing survivors to CVD death only, although the significance was reduced as there were fewer patients. Thickness % and the Area % showed a non-significant difference (p=0.66, p=0.60) to the CVD group, but were both marginally significant in the combined CVD/cancer group (p=0.03, p=0.04).

The number of calcified deposits (NCD) provided the highest significance and predictive power among the single markers (p<0.001, CVD/cancer). The combined MACD index provided the highest significance (down to p=0.00000008 un-adjusted) for all groups of deceased (except for other-cause deaths).

After adjusting the AC24 for the influence of NCD, no significant difference was found between survivors and deceased (p=0.34). The NCD, however, still provided a significant difference (p=0.003) after adjusting for the AC24. The only markers that maintained significance after adjustment by the AC24 or NCD were the MAD factor (p=0.03 and p=0.01 respectively) and Area % (p=0.003 and p=0.02 respectively). The Area % lost significance after adjustment by both NCD and MAD factor (p=0.53).

The NCD marker and the combined MACD index exhibited had odds ratios of CVD/cancer mortality of 11.6 and 19.9 respectively. In comparison, the multivariate risk SCORE card and the Framingham point score yielded the statistically significant lower OR of 5.0 and 5.2, respectively. Combining the AC markers with metabolic/physical markers did not significantly improve the odds ratios as shown below. However, triglycerides generally improve all results but the MACD index.

FIG. 5 compares the markers, where the NCD exhibits a significantly higher odds ratio than the AC24 score (p=0.04). The MACD index was significantly higher than any other marker (SCORE p=0.02, Framingham p=0.02, AC24 p=0.0004, Area % p-0.009, Triglycerides p=0.009. Total cholesterol p=0.0002) except the NCD (p=0.37). Stratification into only CVD death yielded similar results with MACD index odds-ratio at 21, which was significantly higher than any other marker (SCORE OR 4.8 m, p=0.04; Framingham OR 2.8, p=0.006; AC24 OR 3.1, p=0.007; Area % OR 2.4, p=0.004; Triglyceride OR 5.1, p=0.06; Total cholesterol OR 4.2, p=0.03).

The MACD index separated CVD death from survivors (area under ROC-curve 0.85) better than the metabolic physical markers (SCORE 0.80, Framingham 0.78, Triglyceride 0.68, Total cholesterol 0.76). Combination of the MACD index with any of the before mentioned scores resulted in an area under the ROC curve of up to 0.89 when combined with triglyceride concentrations, and hereby provided the largest improvement in the low risk range.

In general, all of the direct AC markers separated survivors from deceased. However, the number of calcified deposits, NCD, provided a superior separation being even more pronounced looking at the odds ratios. This may relate to the fact that even small calcific deposits may in due time develop into vulnerable atherosclerotic lesions. The MACD index, weighting of the NCD with the MAD factor, provided the best separation and highest odds ratios.

The morphological enlargement of plaque described above and used in the MAD factor and MACD index may extract information useful to stratify patients into groups of superior relative risk compared to the atherosclerotic scoring previously used.

The technique described herein is semi-automatic. However, it will be appreciated that the calcific deposit analysis could be fully automated. Specifically, it is possible that the system may be fully automated by using a particle filtering technique in combination with statistical pixel classification to identify and classify the areas of calcification.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’.

1. A computer-implemented method of processing an image of at least part of a blood vessel to derive a measure indicative of the instability of calcific deposits in the blood vessel, said blood vessel containing at least one calcific deposit, which method comprises:
locating and annotating one or more calcific deposits;
using information derived from the annotation of said calcific deposits to calculate a measure reflecting either one or both of a) the aggregate of the deviations from roundness of individual calcific deposits, and b) up to at least a threshold value, the extent to which the separate calcific deposits are spaced from one another.

2. A method as claimed in claim 1, wherein locating and annotating the one or more calcific deposits comprises locating and annotating the boundary of each said calcific deposit and calculating a measure reflecting a combination of a) and b) is obtained by:
calculating the area occupied by the calcific deposits;
expanding the boundary of each said calcific deposit outwards by a distance x corresponding to between 4 mm and 20 mm of a life-size image;
calculating the area occupied by the expanded calcific deposits; and
calculating a comparative index by comparing the area of expanded calcific deposits with the area of unexpanded calcific deposits to derive said measure.

3. A method as claimed in claim 2, wherein the step of expanding the boundary of each said calcific deposit outwards comprises dilating the boundary of each calcific deposit.

4. A method as claimed in claim 3, further comprising dilating the boundary of each said calcific deposit using a circle of radius x.

5. A method as claimed in claim 2, further comprising counting the number of calcific deposits and weighting said comparative index by said number.

6. A method as claimed in claim 1, wherein calculating a measure reflecting b) comprises identifying a convex hull of the calcific deposits and deriving a value representative of the convex hull by calculating one of the perimeter of the convex hull and the area within the convex hull.

7. A method as claimed in claim 6, further comprising calculating a value indicative of the total area of the calcific deposits and dividing the value representative of the convex hull by the total area.

8. A method as claimed in claim 6, further comprising counting the number of calcific deposits and deriving a value indicative of the product obtained by multiplying the number of calcific deposits with the value representative of the convex hull.

9. A method as claimed in claim 1, wherein calculating a measure reflecting a) comprises:
identifying a convex hull of each individual calcific deposit;
deriving a value representative of each convex hull by calculating one of the perimeter of the convex hull or the area within the convex hull and summing the values;
calculating a value indicative of the total area of the calcific deposits; and
dividing the sum of values representative of the convex hulls by the value indicative of the total area of calcific deposits.

10. A method as claimed in claim 1, wherein calculating a measure reflecting a) comprises deriving a value indicative of the result of calculating the ratio of the square of the perimeter to the area for each calcific deposit and summing the ratios.

11. A method as claimed in claim 1, wherein calculating a measure reflecting a) comprises deriving a value indicative of the result of calculating the ratio of the square of the sum of the perimeters of the calcific deposits to the sum of the areas of the calcific deposits.

12. A method as claimed in claim 1, wherein calculating a measure reflecting a) or b) further comprises calculating a value indicative of a fractal dimension of the calcific deposits.

13. A method as claimed in claim 12, further comprising using a box counting method to calculate a value indicative of the fractal dimension of the calcific deposits.

14. A method as claimed in claim 1, wherein calculating a measure reflecting b) further comprises calculating a value indicative of the entropy of the calcific deposits.

15. A method as claimed in claim 1, wherein calculating a measure reflecting b) further comprises calculating a value indicative of the sum of the distances between calcific deposits.

16. A method as claimed in claim 1, wherein the blood vessel is an artery.

17. A method as claimed in claim 1, wherein the blood vessel is an aorta.
